(19) United States
(12) Patent Application Publication
(10) Pub. No.: US 2004/0216749 A1
(43) Pub. Date: Nov. 4, 2004

(54) Vasomodulation during glaucoma surgery

(76) Inventor: Hosheng Tu, Newport Coast, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/763,569

(22) Filed: Jan. 23, 2004

Related U.S. Application Data
(60) Provisional application No. 60/442,098, filed on Jan. 23, 2003.

(51) Int. Cl. A61F 9/007

(52) U.S. Cl. 128/898; 623/905

(57) ABSTRACT

A method of minimizing blood reflux from an episcleral vein during an ab interno eye surgery includes advancing an applicator from an anterior chamber through trabecular meshwork toward Schlemm's canal, and administering a vasoconstrictive agent to about Schlemm's canal for minimizing the blood reflux. The vasoconstrictive agent may be an alpha-adrenergic agonist.

### CROSS-REFERENCE TO RELATED APPLICATIONS

This application claims the priority benefit of U.S. Provisional Application No. 60/442,098, filed Jan. 23, 2003, the entirety of which is hereby incorporated by reference.

### FIELD OF THE INVENTION

The present invention relates to the use of topical vasomodulating agents for ocular surgery.

### BACKGROUND OF THE INVENTION

About two percent of people in the United States have glaucoma. Glaucoma is a group of eye diseases that causes pathological changes in the optic disk and corresponding visual field loss resulting in blindness if untreated. Intraocular pressure elevation appears to be a major etiologic factor in glaucoma.

In glaucomas associated with an elevation in eye pressure, the source of resistance to outflow is in the trabecular meshwork. The tissue of the trabecular meshwork allows aqueous humor, or “aqueous,” to enter Schlemm's canal, which then empties into aqueous collector channels in the posterior wall of Schlemm's canal and then into aqueous veins. Aqueous is a transparent liquid that fills the region between the cornea at the front of the eye and the lens. Aqueous humor is constantly secreted by the ciliary body around the lens, so there is a continuous flow of the aqueous humor from the ciliary body to the eye's front chamber. The eye's pressure is determined by a balance between the production of aqueous and its exit through the trabecular meshwork (major route) or via uveal scleral outflow (minor route). The trabecular meshwork is located between the outer rim of the iris and the internal periphery of the cornea. The portion of the trabecular meshwork adjacent to Schlemm's canal causes most of the resistance to aqueous outflow (juxtacanilicular meshwork).

Glaucoma is grossly classified into two categories: closed-angle glaucoma and open-angle glaucoma. Closed-angle glaucoma is caused by closure of the anterior angle by contact between the iris and the inner surface of the trabecular meshwork. Closure of this anatomical angle prevents normal drainage of aqueous humor from the anterior chamber of the eye. Open-angle glaucoma is any glaucoma in which the angle of the anterior chamber remains open, but the exit of aqueous through the trabecular meshwork is diminished. The exact cause for diminished filtration is unknown for most cases of open-angle glaucoma. However, there are secondary open-angle glaucomas that may involve edema or swelling of the trabecular spaces (e.g., from steroid use), abnormal pigment dispersion, or diseases such as hyperthyroidism that produce vascular congestion.

Current therapies for glaucoma are directed at decreasing intraocular pressure. Treatment generally begins with medical therapy, using eyedrops or pills that reduce the production of aqueous humor or increase the outflow of aqueous. However, various drug therapies for glaucoma are sometimes associated with significant side effects, such as headache, blurred vision, allergic reactions, death from cardiopulmonary complications, and potential interactions with other drugs. When drug therapy fails, surgical therapy is used. Surgical therapy for open-angle glaucoma consists of laser trabeculoplasty, trabeculotomy, trabeculectomy, and aqueous shunting implants after failure of trabeculectomy or if trabeculectomy is unlikely to succeed. Trabeculectomy is a major surgery that is most widely used and is augmented with topically applied anticancer drugs such as 5-flurouracil or mitomycin-c to decrease scarring and increase surgical success.

Approximately 100,000 trabeculectomies are performed on Medicare age patients per year in the United States. This number would increase if the morbidity associated with trabeculectomy could be decreased. The current morbidity associated with trabeculectomy consists of failure (10-15%), infection (a lifelong risk about 2-5%), choroidal hemorrhage (1%, a severe internal hemorrhage from pressure too low resulting in visual loss), cataract formation, and hypotony maculopathy (potentially reversible visual loss from pressure too low).

If it were possible to bypass the local resistance to outflow of aqueous at the point of the resistance and use existing outflow mechanisms, surgical morbidity would greatly decrease. The reason for this is that the episcleral aqueous veins have a backpressure that would prevent the eye pressure from going too low. This would virtually eliminate the risk of hypotony maculopathy and choroidal hemorrhage. Furthermore, visual recovery would be very rapid and risk of infection would be very small (a reduction from 2-5% to 0.05%). Because of these reasons surgeons have tried for decades to develop a workable surgery for the trabecular meshwork.

Other techniques have been tried, including goniotomy/trabeculotomy, and other mechanical disruption of the trabecular meshwork, such as trabeculopuncture, goniophotoablation, laser trabecular ablation and goniocurretage. They are briefly described below.

Goniotomy/Trabeculotomy: Goniotomy and trabeculotomy are simple and directed techniques of microsurgical dissection with mechanical disruption of the trabecular meshwork. These initially had early favorable responses in the treatment of open-angle glaucoma. However, long-term review of surgical results showed only limited success in adults. In retrospect, these procedures probably failed secondary to repair mechanisms and a process of “filling in.” The filling in is the result of a healing process that has the detrimental effect of collapsing and closing in of the created opening throughout the trabecular meshwork. Once the created openings close, the pressure builds back up and the surgery fails.

Trabeculopuncture: Q-switched Neodymium (Nd):YAG lasers also have been investigated as an optically invasive technique for creating full-thickness holes in trabecular meshwork. However, the relatively small hole created by this trabeculopuncture technique exhibits a filling in effect and fails.

Goniophotoablation/Laser Trabecular Ablation: Goniophotoablation is disclosed by Berlin in U.S. Pat. No. 4,846,172, and describes the use of an excimer laser to treat glaucoma by ablating the trabecular meshwork. This was not demonstrated by clinical trial to succeed. Hill et al. used an Erbium:YAG laser to create full thickness holes through trabecular meshwork (Hill et al., Lasers in Surgery and Medicine 11:341-346, 1991). This technique was investigated in a primate model and a limited human clinical trial at the University of California, Irvine. Although morbidity was zero in both trials, success rates did not warrant further human trials. Failure was again from filling in of created defects in trabecular meshwork by repair mechanisms.

Goniocurretage: This is an ab-interno (from the inside) mechanical disruptive technique. This uses an instrument similar to a cyclodialysis spatula with a microcurrette at the tip. Initial results are similar to trabeculotomy that fails secondary to repair mechanisms and a process of filling in.

Although trabeculectomy is the most commonly performed filtering surgery, viscocanalostomy (VC) and nonpenetrating trabeculectomy (NPT) are two new variations of filtering surgery. These are ab-extemo (from the outside), major ocular procedures in which Schlemm's canal is surgically exposed by making a large and very deep scleral flap. In the VC procedure, Schlemm's canal is cannulated and viscoelastic substance injected (which dilates Schlemm's canal and the aqueous collector channels). In the NPT procedure, the inner wall of Schlemm's canal is stripped off after surgically exposing the canal.

Trabeculectomy, VC, and NPT are performed under a conjunctival and scleral flap, such that the aqueous humor is drained onto the surface of the eye or into the tissues located within the lateral wall of the eye. Normal physiological outflows are not used. These surgical operations are major procedures with significant ocular morbidity. When Trabeculectomy, VC, and NPT are thought to have a low chance for success, a number of implantable drainage devices have been used to ensure that the desired filtration and outflow of aqueous humor through the surgical opening will continue. The risk of placing a glaucoma drainage implant also includes hemorrhage, infection, and postoperative double vision.

The above treatment modalities and variations thereof have numerous disadvantages and generally only moderate success rates. They involve substantial trauma to the eye and require great surgical skill by creating a hole over the full thickness of the sclera/comea into the subconjunctival space. Furthermore, normal physiological outflow pathways are not used. The procedures are generally performed in an operating room, generating a facility fee and anesthesiologist's professional fee, and have a prolonged recovery time for vision. The complications of filtration surgery have inspired ophthalmic surgeons to look at other approaches to lowering intraocular pressure.

Therefore, there is a great clinical need for the treatment of glaucoma by a method that would be faster, safer and less expensive than currently available modalities. Trabecular bypass surgery is an innovative surgery that uses a micro stent, shunt, or other implant to bypass diseased trabecular meshwork alone at the level of trabecular meshwork and use or restore existing outflow pathways. The object of the present invention is to provide means and methods for treating elevated intraocular pressure by implanting a trabecular stent in an ab interno manner with topically administered vasoconstrictive agents to facilitate stent implantation by causing vasoconstriction and enhanced analgesia.

### SUMMARY OF THE INVENTION

U.S. Pat. No. 6,638,239, the entirety of which is incorporated herein by reference, discloses trabecular bypass surgery procedures that bypass diseased trabecular meshwork at the level of the trabecular meshwork and utilize existing outflow pathways. However, during the trabecular bypass procedures, blood reflux from episcleral veins can tend to blur visualization and compromise surgical precision. Some embodiments of the invention involve topically administering a vasoconstrictive agent to mitigate undesired blood reflux.

In this description, “ab interno” glaucoma surgery means surgery that involves advancing an implant from the anterior chamber of an eye through trabecular meshwork toward Schlemm's canal. “Ab extemo” surgery involves insertion of an implant through the sclera or corneoscleral junction into Schlemm's canal and/or trabecular meshwork before or without entering the anterior chamber.

In some embodiments, trabecular bypass surgery ab interno is used to bypass diseased trabecular meshwork at the level of trabecular meshwork and use existing outflow pathways.

In some embodiments a trabecular stent or implant is implanted at the level of trabecular meshwork for transporting aqueous humor from the anterior chamber to Schlemm's canal.

In another embodiment, a method is provided for topically administered an alpha agonist for facilitating a trabecular bypass surgery and subsequent trabecular stent implantation by decreasing pain and/or minimizing reflux of blood.

In some embodiments, a method is provided for topically administering a vasoconstrictive agent or a pharmaceutically acceptable salt thereof for facilitating trabecular bypass surgery and subsequent trabecular stent implantation or other ocular surgery by administering a therapeutically effective amount of the compound.

Some embodiments comprise a method of minimizing blood reflux from an episcleral vein or reducing pain during eye surgery, comprising advancing an implant from an anterior chamber through trabecular meshwork toward Schlemm's canal, and administering a vasoconstrictive agent to eye tissue to decrease blood flow through an episcleral vein or reduce pain.

In some embodiments, the vasoconstrictive agent is an alpha agonist. In some embodiments, the administering is topical. Some embodiments further comprise administering a second agent to eye tissue. In some embodiments, the second agent is tetracaine.

In some embodiments, the second agent is brimonidine. In some embodiments, the vasoconstrictive agent is an eye solution with a pH between 4 and 8. In some embodiments, the vasoconstrictive agent is an active ingredient in an eye solution at a concentration between 0.01 and 2 weight percent.

In some embodiments, the implant is configured to be placed through the trabecular meshwork such that a proximal terminal of the implant is exposed to the anterior chamber and a distal terminal is exposed to Schlemm's canal.

Some embodiments include a method of enhancing penetration of a poorly absorbing eye medicine, comprising co-administering to the eye of a mammal a therapeutic amount of vasoconstrictor and a therapeutic amount of said poorly absorbing eye medicine.

In some embodiments, the vasoconstrictor is selected from the group consisting of alpha-1 agonist, alpha-2 agonist, alpha-3 agonist, and beta-adrenergic antagonist. In some embodiments, the poorly absorbing eye medicine is tetracaine. In some embodiments, the poorly absorbing eye medicine is brimonidine.

Some embodiments include a method of minimizing blood reflux from an episcleral vein or reducing pain during eye surgery, comprising placing a glaucoma implant into an eye of a mammal, and administering a vasoconstrictive agent to the eye.

Some embodiments of the invention relate to a method of enhancing penetration of a poorly absorbing eye medicine, comprising co-administering to the eye of a mammal a therapeutic amount of alpha-adrenergic agonist (“alpha agonist”) and a therapeutic amount of the poorly absorbing eye medicine. The poorly absorbing eye medicine can comprise tetracaine.

Some embodiments of the invention provide a method of minimizing pain during an ab interno eye surgery, comprising: providing ab interno surgery by advancing an applicator and/or implant from the anterior chamber of an eye through trabecular meshwork toward Schlemm's canal; administering a pain reducing agent to the eye, about Schlemm's canal for minimizing the pain, wherein the pain reducing agent can be an alpha agonist.

### DETAILED DESCRIPTION OF THE PREFERRED EMBODIMENT

Alpha agonists can reduce pain, mainly through an alpha-2 mechanism. Topical application can minimize systemic side effects. These agents have good ocular absorption (penetration), and the added penetration and decreased washout of blood due to local vasoconstriction help to improve absorption of other topical agents with poor penetration, such as tetracaine. This aids in procedures that are minimally invasive, such as trabecular bypass surgery (TBS) or other ocular surgery. In addition, the vasoconstrictive effect also assists in constricting aqueous veins and minimizing reflux of episcleral blood during trabecular stent implantation with clear visualization.

Pharmaceutical compositions having an adrenergic compound or compounds as the active ingredient are useful for treating glaucoma, chronic pain, nasal congestion, high blood pressure, congestive heart failure and inducing anesthesia.

Some embodiments of the invention relate to devices and methods for treating elevated intraocular pressure by implanting a trabecular stent in an ab interno manner with a topically administered vasoconstrictive agent or system to facilitate stent implantation by causing vasoconstriction resulting in reduced blood reflux from episcleral veins so as to maintain the incision areas visually clear. Vasoconstrictive agents that may be used include those of the alpha agonist system, the angiotensin-aldosterone system, the arginine-vasopressin system, the sympathetic nerve system (both alpha agonists and beta antagonists), sumatriptan, endothelins (such as Endothelin-1), prostaglandins, and the like.

Vasoconstrictor agents of the invention may further include, but are not limited to, catecholamines, e.g., epinephrine, norepinephrine, and dopamine, as well as metaraminol, phenylephrine, methoxamine, mephentermine, methysergide, ergotamine, ergotoxine, dihydroergotamine, sumatriptan and analogs, and alpha-1 and alpha-2 adrenergic agonists, such as, e.g., clonidine, guanfacine, guanabenz and dopa (i.e., dihyrdoxyphenylalanine), methyldopa, ephedrine, amphetamine, methamphetamine, methylphenidate (Ritalin), ethylnorepinephrine, pemoline, and other sympathomimetic agents, including active metabolites, derivatives and mixtures of any of the foregoing.

Alpha Agonist System

Vasoconstriction of blood vessels is achieved by stimulation of the alpha receptors in the smooth muscle cells of the blood vessel wall. Vasoconstriction is desirable in some clinical situations locally to reduce regional blood flow. The vasoconstrictor properties would substantially reduce blood flow in blood vessels and can be used to prevent hemorrhaging associated with external or internal injuries without the risk of thrombosis. These compounds may also be used as surgical adjuncts to reduce the bleeding from incisions at any anatomical location, such as in trabecular bypass surgery procedures. The alpha-1 adrenergic receptors, found in the smooth muscle cells of the peripheral vasculature of the coronary arteries, skin, uterus, intestinal mucosa, and episcleral veins, mediate vasoconstriction. These receptors serve as postsynaptic activators of vascular and intestinal smooth muscle. Their activation results in either decreased or increased tone, depending upon the effector organ. The response in resistance and capacitance blood vessels is constriction.

The agents may be characterized by their activity, i.e. as stimulating agents (agonists) or blocking agents (antagonists), and by the specific type of adrenoceptors upon which they act. The two main families of adrenergic receptor are termed alpha-adrenergic receptors and beta-adrenergic receptors, and each of these two families is known to have subtypes, which are designated by letters of the alphabet, such as alpha-1 (with subtype alpha-1A, alpha-1B, and so forth) and alpha-2 (with subtype alpha-2A, alpha-2B, and so forth). Adrenergic agents can exert their activity by interaction with adrenal receptors (adrenoceptors). The concept of post-junctional alpha-2 adrenoceptors mediating prazosin-resistant vasoconstriction has been proposed by Timmermans, et al., Nauyn-Schmiedeberg's Arch. Pharmacol., 310, 189 (1979), and Ruffolo, Pharm. Biochem. and Behav., 22, 827 (1985).

U.S. Pat. No. 6,329,369, issued on Dec. 11, 2001, the entire contents of which are incorporated herein by reference, discloses methods of treating glaucoma or elevated pressure and other diseases with reduced side effects by treating a mammal in need thereof with an agonist of the alpha-2B or alpha-2B/2C adrenergic receptors.

Treatment of glaucoma or any other indications known or discovered to be susceptible to treatment by adrenergic compounds will be effected by administration of therapeutically effective dose of one or more compounds in accordance with the instant invention. A therapeutic concentration will be that concentration which effects reduction of the particular condition, or retards its expansion. In certain instances, the drug potentially could be used in a prophylactic manner to prevent onset of a particular condition. A given therapeutic concentration will vary from condition to condition and in certain instances may vary with the severity of the condition being treated and the patient's susceptibility to treatment. Accordingly, a given therapeutic concentration will be best determined at the time and place through routine experimentation.

U.S. Pat. No. 5,021,410 issued on Jun. 4, 1991, and U.S. Pat. No. 5,180,721 issued on Jan. 19, 1993, the entire contents of which are incorporated herein by reference, disclose methods and pharmaceutical formulations of alpha-2 agonists and alpha-3 antagonists that are useful in lowering intraocular pressure (IOP) and treatment of intraocular hypertension. Co-administration of a therapeutic amount of alpha-2 agonist with a potentiating amount of alpha-3 agonist can be effective in lowering IOP and treatment of intraocular hypertension.

It should be noted that alpha agonists and alpha antagonists appear to preferentially stimulate or block each type of adrenoceptor with varying degrees of specificity rather than exclusively stimulating or blocking one adrenoceptor sub-type. For example, clonidine preferentially stimulates alpha-2 adrenoceptor≧alpha-3 adrenoceptor>>alpha-1 adrenoceptor and is therefore characterized as an alpha-2 agonist. Topically administered alpha agonists that are efficacious in lowering IOP are part of current ocular hypertension therapy, including glaucoma therapy. In some embodiment of the present invention, it is provided a topically administered alpha agonist for facilitating a trabecular bypass surgery (TBS) and subsequent trabecular stent implantation. These agents have good ocular penetration and the added penetration can be beneficial to other topical agents with poor penetration, such as tetracaine and the like. In one embodiment of the present invention, tetracaine is mixed physically with an alpha agonist for enhanced ocular penetration (absorption) for ophthalmology treatment.

Furthermore, U.S. Pat. No. 5,215,991, issued on Jun. 1, 1993, the entire contents of which are incorporated herein by reference, discloses methods and pharmaceutical compositions of alpha-2 agonists and Na⁺/H⁺ exchange inhibitors which are useful in lowering intraocular pressure (IOP) and treatment of intraocular hypertension. Co-administration of a therapeutic amount of alpha-2 agonist with a potentiating amount of Na⁺/H⁺ exchange inhibitor is effective in lowering IOP and treatment of intraocular hypertension. Co-administration refers to administering compounds serially, or at substantially the same time or simultaneously. Co-administration includes administering the compounds separately but at substantially the same time, or administering them at the same time in one pharmaceutical preparation. Serial administration includes administering the compounds of the present invention one after the other wherein the time between administrations of the compounds is about one hour or less.

Additionally, phenylephrine is considered a potent pure alpha agonist drug that increases venous as well as arterial constriction. Phenylephrine is used intravenously in small doses of 1 μg/kg to cause systemic vasoconstriction and elevation of blood pressure. It is also used regionally to cause vasoconstriction when injected with local anesthetic agents to provide prolonged nerve conduction block. Phenylephrine has been found to provide excellent decongestion of the nasal mucosa by exerting its alpha-1 mediated vasoconstricting effect on the mucosal blood vessels. This directly opposes histamine-mediated vasodilation and reduces mucosal edema and vascularity. Other agents that have been used for this effect are ephedrine and cocaine.

Vasoconstrictors of the invention may comprise serotonin, thromboxane A2, endothelin-1, angiotensin-II, and the like. Thromboxane A2 is formed from endoperoxides by the sequential actions of the enzymes cyclooxygenase and thromboxane synthetase in platelet microsomes. Thromboxane A2 is readily generated by platelets and is a potent vasoconstrictor, by virtue of its capacity to produce platelet aggregation.

The renin-angiotensin-aldosterone system (RAAS) is one of the hormonal mechanisms involved in regulating pressure/volume homeostasis and also in the development of hypertension. Activation of the renin-angiotensin-aldosterone system begins with renin secretion from the juxtaglomerular cells in the kidney and culminates in the formation of angiotensin II, the primary active species of this system. This octapeptide, angiotensin II, is a potent vasoconstrictor and also produces other physiological effects such as stimulating aldosterone secretion, promoting sodium and fluid retention, inhibiting renin secretion, increasing sympathetic nervous system activity, stimulating vasopressin secretion, causing positive cardiac inotropic effect and modulating other hormonal systems. In this system, renin, a proteolytic enzyme formed in the granules of the juxtaglomerular apparatus cells catalyzes the conversion of angiotensinogen (a plasma protein) into angiotensin I, a decapeptide.

This inactive product is then cleaved by a converting enzyme, termed angiotensin converting enzyme (ACE) mainly in the lung, but also in the kidney and brain, to an octapeptide, angiotensin II, which is a potent vasoconstrictor and also stimulates the release of aldosterone. Aldosterone is an adrenal cortex hormone that promotes the retention of salt and water by the kidneys and thus increases plasma volume, resulting in an increase in blood pressure. Angiotensin II also stimulates the release of norepinephrine from neural cells and interacts with specific receptors on blood vessels, thereby resulting in an increase in calcium and vasoconstriction. Another mechanism by which angiotensin II induces vasoconstriction is by interacting with specific receptors on blood vessels, thereby resulting in an opening of calcium channels and an increase in calcium, resulting in vasoconstriction. The hormone angiotensin II (AII) produces numerous biological responses (e.g. vasoconstriction) through stimulation of its receptors on cell membranes.

The vascular architecture is maintained or remodeled in response to the changes in the balance of paracrine factors. One of the substances that participate in vascular homeostasis is endothelium derived nitric oxide (NO). NO is synthesized from the amino acid L-arginine by NO synthase. NO relaxes vascular smooth muscle and inhibits its proliferation. In addition, NO inhibits the interaction of circulating blood elements with the vessel wall. NO activity is reduced in hypercholesterolemia and after vascular injury. We have shown that administration of the NO precursor (L-arginine) has been shown to restore vascular NO activity in animals and in humans with endothelial vasodilator dysfunction due to hypercholesterolemia, atherosclerosis, or restenosis. Chronic enhancement of NO activity (by oral administration of L-arginine) is associated with a significant reduction in intimal thickening due to hypercholesterolemia and/or vascular injury.

Serotonin receptor agonists, such as sumatriptan, are thought to constrict dilated arteries. Endothelin has been shown to affect the cells in the heart both in vivo and in vitro. In vivo endothelin is present in both atrial and ventricular myocardium in healthy and failing hearts and enhances myocardial inotropic activity, vascular smooth muscle proliferation, and coronary vasoconstriction. Endothelin with functional vasoconstriction for acute problems narrows radius of a vascular vessel to increase pressure. Endothelin is released from the endothelial tissue in response to pressures encountered and its goal is to protect the vessels and tissue at all costs. It is present in the endothelial cells of most blood vessels, and is released in response to bleeding too. Endothelin-1 is a 21 amino acid peptide that is a potent venous and arterial vasoconstrictor.

U.S. Pat. No. 6,090,825, issued on Jul. 18, 2000, the entire contents of which are incorporated herein by reference, discloses oxazole derivatives as antagonists of alpha-1C adrenergic receptors. Compounds that modulate alpha-1-adrenergic subtype response have additionally been implicated as useful for treatment of conditions such as hypertension. Piperazinyl oxazole compounds have been described for use as anti-inflammatory, analgesic and antihistamine agents.

U.S. Pat. No. 4,440,769 issued on Apr. 3, 1984, the entire contents of which are incorporated herein by reference, discloses that the adrenergic nervous system plays a primary role in the neurogenic regulation of the cardiovascular system. The sympathetic outflow to the heart and peripheral vessels originates from the vasomotor center and travels along descending neuronal pathways interrupted by synapses, the switching units which transmit the neurological signal from higher to lower neurons and from nerve endings to cells of the effector organ. Transmission of the neurological signal across synapses is mediated chemically by a neurotransmitter that is stored in the vesicles of nerve endings. Upon arrival of the neurological signal, regulated quantities of neurotransmitter are released into the synapse where it combines with receptor sites in the cellular membrane of the next neuron or effector organ, and excites the receptor cell to propagate the neurological signal or to produce an effect in an effector organ.

The principal natural neurotransmitters specific to the adrenergic nervous system are norepinephrine and epinephrine, which mediate neurological transmission in some central noradrenergic neurons in the vasomotor center and elsewhere in the brain as well as peripherally in so-called postganglionic sympathetic neurons. Receptors for norepinephrine have been recognized to be proteins bound to membranes of effector cells. These receptors control the function of the effector cell, and through it the function of a whole organ or organ systems. Norepinephrine receptors are highly specific for norepinephrine and can discriminate between it and many other transmitters and molecules. However, their discrimination capability is not complete, and other related catecholamines as well as various synthetic agents have been found to bind to norepinephrine receptors.

Through observed responses of various tissues and organs to norepinephrine and related catecholamine-like compounds, it has been found that norepinephrine receptors differ substantially in different tissues where they mediate different functions. In addition, norepinephrine receptors from various tissues have been found to differ in their discriminatory abilities for other compounds. Based on the foregoing and other observations, norepinephrine receptors have been classified into at least two major groups, i.e., the alpha-adrenergic receptors and the beta-adrenergic receptors. In addition, the alpha-groups of receptors have been further divided into the alpha-1 adrenergic receptor sub-group and the alpha-2 adrenergic receptor sub-group. The alpha-1 adrenergic receptors have been characterized as being excitatory in nature, primarily functioning to result in peripheral vascular contraction. On the other hand, the alpha-2 adrenergic receptors have been characterized as being inhibitory in nature, primarily functioning to inhibit transmitter release through inhibition of adenylate cyclase activity.

Inasmuch as the different groups and sub-groups of adrenergic receptors mediate different functions in different bodily tissues and organs, it is highly desirable to obtain chemical compounds or entities that are highly selective for limited types of receptor sites. In this manner, isolated symptoms can be effectively treated, without affecting other unrelated tissues and organs, by selectively agonizing or antagonizing a particular sub-group of receptor sites. One compound which has been found to selectively antagonize alpha-1 adrenergic receptor sites is known generically as prazosin, wherein prazosin has also been used as a model alpha-1 antagonist in the evaluation of other compounds for alpha-1 agonistic or antagonistic activity.

Compound Composition

The alpha agonists (“compounds”) can be incorporated into various types of ophthalmic formulations for topical delivery to the eye. They may be combined with ophthalmologically acceptable preservatives, surfactants, viscosity enhancers, penetration enhancers, buffers, sodium chloride, and water to form aqueous, sterile ophthalmic suspensions or solutions. Ophthalmic solution formulations may be prepared by dissolving the compound in a physiologically acceptable isotonic aqueous buffer. Further, the ophthalmic solution may include an ophthalmologically acceptable surfactant to assist in dissolving the compound. The ophthalmic solutions may contain a thickener, such as, hydroxymethylcellulose, hydroxyethylcellulose, hydroxypropylmethylcellulose, methylcellulose, polyvinyl-pyrolidone, or the like, to improve the retention of the formulation in the conjunctival sac. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-940, or the like, according to the published formulations for analogous ophthalmic preparations; preservatives and tonicity agents can be incorporated. Preparation of such topical formulations are well described in the art of pharmaceutical formulations as exemplified, for example, by Remington's Pharmaceutical Science, Edition 17, Mack Publishing Company, Easton, Pa.

If dosed topically, the compounds are preferably formulated as topical ophthalmic suspensions or solutions, with a pH of about 4 to 8, preferably about 7. The compounds will normally be contained in these formulations in an amount 0.001% to 5% by weight, but preferably in an amount of 0.01% to 2% by weight. Thus, for topical presentation, 1 to 2 drops of these formulations would be delivered to the surface of the eye according to the routine discretion of a skilled clinician.

The preferred compound, alpha agonist, may be mixed with an IOP-lowering agent for treating glaucoma patients. The IOP-lowering agents useful in the present invention include all presently known IOP-lowering pharmaceuticals, including, but not limited to, miotics (e.g., pilocarpine, carbachol, and acetylcholinesterase inhibitors); alpha and alpha/beta adrenergic agonists (e.g., epinephrine, dipivalylepinephrine, para-amino clonidine and brimonidine); beta-blockers (e.g., betaxolol, S-betaxolol, levobunolol, carteolol, and timolol); prostaglandins and their analogues and derivatives, such as, compounds disclosed in U.S. Pat. Nos. 4,599,353; 5,093,329; and 5,321,128; and carbonic anhydrase inhibitors (e.g., acetazolamide, methazolamide, and ethoxzolamide, and compounds disclosed in U.S. Pat. Nos. 5,153,192; 5,240,923; 5,378,703; and 4,797,413) and ocular hypertensive lipids, such as those compounds (neutral replacement of the carboxylic acid group of prostaglandin F2α e.g. AGN 192024) described in IOVS, Mar. 15, 1998, Vol. 39, No. 4; WO 97/30710, U.S. Pat. Nos. 5,238,961; 5,262,437; 5,328,933; 5,352,708; 5,312,842; 5,552,434; 5,545,665; 5,688,819. The preferred IOP-lowering agents are: timolol, betaxolol, S-betaxolol levobunolol, carteolol, pilocarpine, carbachol, epinephrine, dipivalyl epinephrine-α methyl dipivalylepinephrine, brinzolamide, dorzolamide, unoprostone, latanoprost, travoprost, apraclonidine, and brimonidine.

The alpha agonists with one or more IOP-lowering agents will be administered topically at a concentration of between about 0.001 and 5.0 wt %, preferably, about 0.01 to 2.5 wt %, but preferably about 0.001-0.005 wt % for prostaglandins.

In addition to alpha agonists, the additional active ingredient(s) that can be included in the compositions of the present invention include all ophthalmic, dermatological, otic, or nasal agents that can be topically applied. For example, such ophthalmic agents include (but are not limited to): anti-glaucoma agents, such as beta-blockers (e.g., betaxolol and timolol), muscarinics (e.g., pilocarpine), prostaglandins, carbonic anhydrase inhibitors (e.g., acetazolamide, methazolamide and ethoxzolamide), dopaminergic agonists and antagonists, and alpha adrenergic receptor agonists, such as para-amino clonidine (also known as apraclonidine) and brimonidine; anti-infectives, such as ciprofloxacin; non-steroidal and steroidal anti-inflammatories, such as suprofen, ketorolac, dexamethasone, rimexolone and tetrahydrocortisol; proteins; growth factors, such as EGF; and anti-allergic agents, such as cromolyn sodium, emedastine, and olopatadine. Compositions of the present invention may also include combinations of active ingredients.

The compositions of the present invention can also include other components, for example, pharmaceutically acceptable buffers; tonicity agents; comfort-enhancing agents; solubilizing aids; pH adjusting agents; antioxidants; and stabilizing agents. The compositions may also contain additional preservatives (in conjunction with the cationic preservatives addressed above). As will be appreciated by those skilled in the art, the compositions may be formulated in various dosage forms suitable for topical delivery, including solutions, suspensions, emulsions, and gels.

During TBS, clear visualization is critical for a surgeon to see where the trabecular stent penetrates through trabecular meshwork into Schlemm's canal. Once the device cuts into the tissue, blood reflux from the aqueous veins of the existing outflow pathways may blurring the surgical field. The alpha agonists have the vasoconstrictive properties that may constrict aqueous veins and minimize reflux of blood through or around the stent. In some embodiment of the present invention, other topical vasoconstrictive agent(s) can be used for facilitating trabecular bypass surgery (TBS) and subsequent trabecular stent implantation by decreasing pain and/or minimizing reflux of blood. Some embodiments of the present invention provide a method for topically administered a vasoconstrictive agent or a pharmaceutically acceptable salt thereof for facilitating a trabecular bypass surgery and subsequent trabecular stent implantation or other ocular surgery by administering a therapeutically effective amount of the compound.

During TBS, it is desirable to reduce pain, mainly through an alpha-2 mechanism. Some embodiments of the invention provide a method of minimizing pain during an ab interno eye surgery, comprising: providing the ab interno surgery by advancing an applicator from an anterior chamber through trabecular meshwork toward Schlemm's canal; administering a pain reducing agent to about Schlemm's canal for minimizing the pain, wherein the pain reducing agent is an alpha agonist.

Some embodiments of the invention provide a method of enhancing penetration of a poorly absorbing eye medicine, comprising co-administering to the eye of a mammal a therapeutic amount of alpha-adrenergic agonist and a therapeutic amount of the poorly absorbing eye medicine, wherein the alpha-adrenergic agonist is selected from a group consisting of alpha-1, alpha-2, alpha-3, and beta-1 adrenergic agonist. The poorly absorbing eye medicine can comprise tetracaine, brimonidine, and the like.

From the foregoing description, it should now be appreciated that a novel method for topical eye application during a trabecular bypass surgery for the treatment of glaucoma has been disclosed. While the invention has been described with reference to a specific embodiment, the description is illustrative of the invention and is not to be construed as limiting the invention. Various modifications and applications may occur to those who are skilled in the art, without departing from the true spirit and scope of the invention.

What is claimed is:

1. A method of minimizing blood reflux from an episcleral vein or reducing pain during eye surgery, comprising:
placing a glaucoma implant into an eye of a mammal; and
administering a vasoconstrictive agent to the eye to reduce blood flow through the episcleral vein or reduce pain.

2. A method of minimizing blood reflux from an episcleral vein or reducing pain during eye surgery, comprising:
advancing an implant from an anterior chamber through trabecular meshwork toward Schlemm's canal; and
administering a vasoconstrictive agent to eye tissue to decrease blood flow through the episcleral vein or reduce pain.

3. The method of claim 1, wherein the vasoconstrictive agent is an alpha agonist.

4. The method of claim 1, wherein the administering is topical.

5. The method of claim 2, further comprising administering a second agent to eye tissue.

6. The method of claim 3, wherein the second agent is tetracaine.

7. The method of claim 3, wherein the second agent is brimonidine.

8. The method of claim 1, wherein the vasoconstrictive agent is an eye solution with a pH between 4 and 8.

9. The method of claim 1, wherein the vasoconstrictive agent is an active ingredient in an eye solution at a concentration between 0.01 and 2 weight percent.

10. The method of claim 1, wherein the implant is configured to be placed through the trabecular meshwork such that a proximal terminal of the implant is exposed to the anterior chamber of the eye and a distal terminal is exposed to Schlemm's canal of the eye.

11. A method of enhancing penetration of a poorly absorbing eye medicine, comprising co-administering to the eye of a mammal a therapeutic amount of vasoconstrictor and a therapeutic amount of said poorly absorbing eye medicine.

12. The method of claim 15, wherein the vasoconstrictor is selected from the group consisting of alpha-1 agonist, alpha-2 agonist, alpha-3 agonist, and beta-adrenergic antagonist.

13. The method of claim 16, wherein the poorly absorbing eye medicine is tetracaine.

14. The method of claim 11, wherein the poorly absorbing eye medicine is brimonidine.
